(19) United States
(12) Patent Application Publication
(10) Pub. No.: US 2022/0369794 A1
(43) Pub. Date: Nov. 24, 2022

(54) MULTIFUNCTIONAL STORAGE BOX

(71) Applicant: Linhui Gui, Jiujiang (CN)

(72) Inventor: Linhui Gui, Jiujiang (CN)

(21) Appl. No.: 17/881,642

(22) Filed: Aug. 5, 2022

(30) Foreign Application Priority Data
Jul. 19, 2022 (CN) 202221849721.6

(51) Int. Cl. A45D 44/06; A45D 20/16; A61L 2/10; A61L 2/26

(52) U.S. Cl. CPC A45D 44/06; A45D 20/16; A61L 2/10; A61L 2/26; A47B 67/02

(57) ABSTRACT

A multifunctional storage box is provided, including a storage box body which is provided with a first storage cavity, a second storage cavity and a third storage cavity; the first storage cavity is provided with a first window used for taking out paper towels; the first window is formed in a front side of the first storage cavity; the second storage cavity is provided with several hanging parts used for hanging toothbrushes; a lower side of the second storage cavity is provided with an avoiding opening for avoiding toothbrushes; and a front side of the third storage cavity is provided with a second window used for allowing an air outlet tube to enter.

### TECHNICAL FIELD

The present disclosure relates to a multifunctional storage box.

### BACKGROUND

The statements here only provide the background information related to the present disclosure, and do not necessarily constitute the prior art.

In a washroom, people will usually use toothbrushes, paper towels (such as facial tissue), hair dryers and other supplies, and some people store these supplies by installing a closet. However, the closet is usually installed by professional installation personnel, at the same time, the cost of installing the closet is also relatively high.

Therefore, it is necessary to study a new technical solution to solve the above problems.

### SUMMARY

In order to solve the defects and shortcomings in the prior art, the present disclosure provides a multifunctional storage box. A storage box can be designed to place paper towels, toothbrushes and a hair dryer, and is particularly suitable for a washroom. Meanwhile, the storage box has the advantages of reasonable layout and space saving due to reasonable arrangement of a first storage cavity, a second storage cavity and a third storage cavity.

In order to achieve the above-mentioned objective, the present disclosure adopts the following technical solution:

A multifunctional storage box includes a storage box body which is provided with a first storage cavity used for placing paper towels, a second storage cavity used for placing toothbrushes, and a third storage cavity used for placing a hair dryer.

The first storage cavity is provided with a first window used for taking out paper towels; the first window is formed in a front side of the first storage cavity.

The second storage cavity is provided with several hanging parts used for hanging toothbrushes; and a lower side of the second storage cavity is provided with an avoiding opening for avoiding toothbrushes.

A front side of the third storage cavity is provided with a second window used for allowing an air outlet tube to enter.

In some embodiments, the first storage cavity is arranged at an upper side of the second storage cavity; and the third storage cavity is arranged at a right side of the first storage cavity.

In some embodiments, the storage box body is further provided with a fourth storage cavity used for placing an atomizer; and a fog outlet is formed in the fourth storage cavity.

In some embodiments, the storage box body includes a bottom shell, a middle plate and a surface cover; the middle plate is arranged on the bottom shell, and is provided with an inner cavity; the surface cover is rotatably connected to the bottom shell, and is located at a front side of the middle plate.

A Printed Circuit Board (PCB) is arranged in the inner cavity; and the PCB is provided with a power line connected to a household power supply and a power supply socket for supplying power.

In some embodiments, the multifunctional storage box further includes a hair dryer placed in the third storage cavity; and the PCB is electrically connected to the hair dryer.

In some embodiments, a rear side of the bottom shell is provided with a wall-mount hole.

In some embodiments, the bottom shell, the middle plate and the surface cover are all made of plastic.

In some embodiments, the second storage cavity is formed between the middle plate and the surface cover; the surface cover is provided with an avoiding window corresponding to the second storage cavity; and the avoiding window is provided with a cover plate in an opening and closing manner.

In some embodiments, an ultraviolet radiator used for disinfecting a toothbrush is arranged in the second storage cavity, and the ultraviolet radiator is electrically connected to the PCB.

Compared with the prior art, the present disclosure has obvious advantages and beneficial effects. Specifically, it can be obtained from the above-mentioned technical solutions that a storage box can be designed to place paper towels, toothbrushes and a hair dryer, and is particularly suitable for a washroom. Meanwhile, the storage box has the advantages of reasonable layout and space saving due to reasonable arrangement of a first storage cavity, a second storage cavity and a third storage cavity.

Secondly, the fourth storage cavity for storing the atomizer is also included, which further enriches the function of the storage box. The power supply socket is also included to improve the practicability of the storage box of the present disclosure.

Furthermore, the ultraviolet radiator is arranged in the second storage cavity, which further ensures the cleanliness and sanitation of personal hygiene products.

In order to describe the structural features and effects of the present disclosure more clearly, the present disclosure is described in detail below in combination with the accompanying drawings and specific embodiments.

### BRIEF DESCRIPTION OF THE DRAWINGS

FIG. 1 is a first three-dimensional schematic diagram (a first use state) of Embodiment I of the present disclosure;

FIG. 2 is a second three-dimensional schematic diagram (a first use state) of Embodiment I of the present disclosure;

FIG. 3 is a first exploded diagram of Embodiment I of the present disclosure;

FIG. 4 is a second exploded diagram of Embodiment I of the present disclosure;

FIG. 5 is a partially exploded diagram of Embodiment I of the present disclosure;

FIG. 6 is a third three-dimensional schematic diagram (a second use state) of Embodiment I of the present disclosure;

FIG. 7 is a fourth three-dimensional schematic diagram (a third use state) of Embodiment I of the present disclosure;

FIG. 8 is an exploded diagram of Embodiment II of the present disclosure.

### REFERENCE SIGNS IN THE DRAWINGS

10: storage box body; 101: bottom shell; 1011: wall-mount hole; 1012: wire avoiding slot; 102: middle plate; 103: surface cover; 1031: avoiding window; 104: supporting column; 1041: vertical column; 1042: supporting disk; 11: first storage cavity; 111: first window; 12: second storage cavity; 121: hanging part; 122: ultraviolet radiator; 123: cover plate; 13: third storage cavity; 131: second window; 14: fourth storage cavity; 141: fog outlet; 40: PCB; 41: power supply socket; 411: toggle switch; 50: hair dryer; 60: tooth mug; 70: atomizer.

### DETAILED DESCRIPTION OF THE EMBODIMENTS

The technical solutions in the embodiments of the present disclosure will be clearly and completely described below in conjunction with the accompanying drawings. Apparently, the described embodiments are only preferred embodiments of the present disclosure.

It should be noted that when an element is referred to as being “fixed” to another element, it can be directly on the other element or an intermediate element may also exist. When an element is considered to be “connected” to another element, it can be directly connected to the other element or an intermediate element may be present at the same time. The terms “perpendicular”, “horizontal”, “left”, “right” and similar expressions used herein are for illustrative purposes only, and are not meant to be the only implementation modes.

Unless otherwise defined, all technical and scientific terms used herein are the same as meanings of general understandings of those skilled in the art of the present disclosure. The terms used in the description of the present disclosure herein are merely to describe the specific implementation modes, not intended to limit the present disclosure. The term “and/or” used herein includes any and all combinations of one or more related listed items.

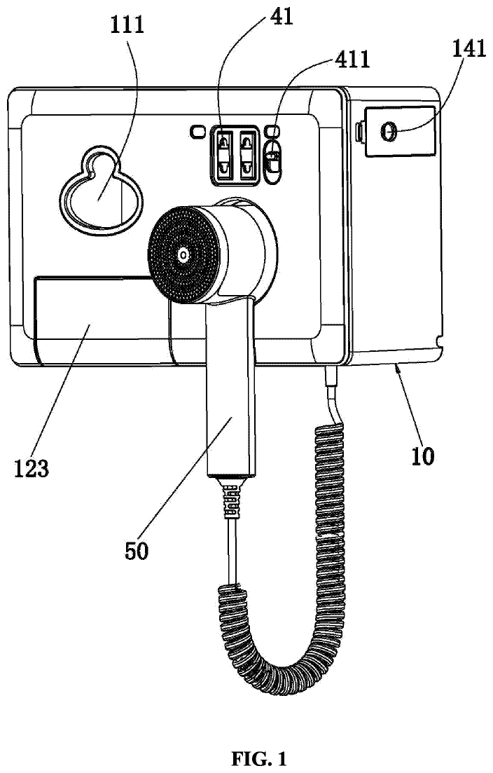

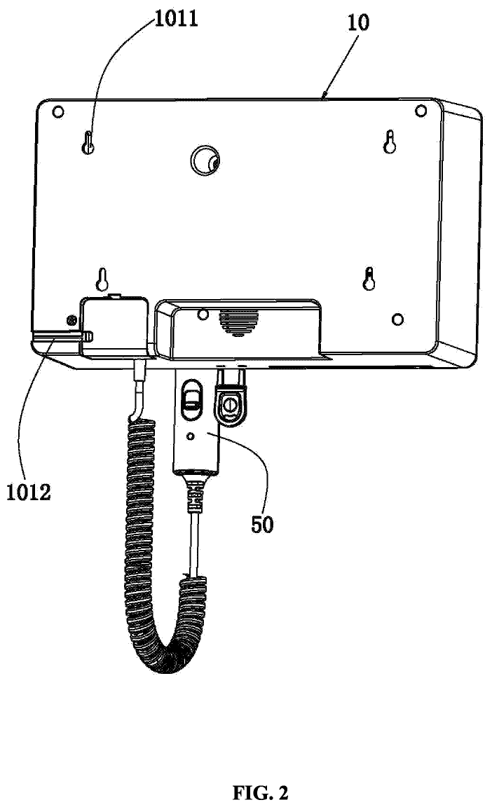

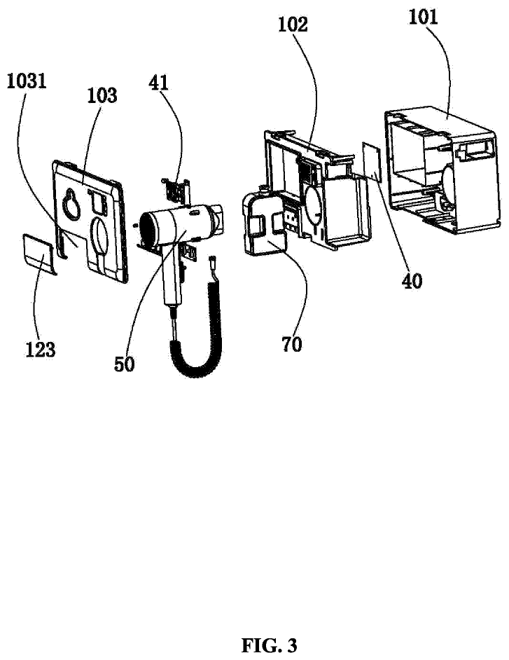

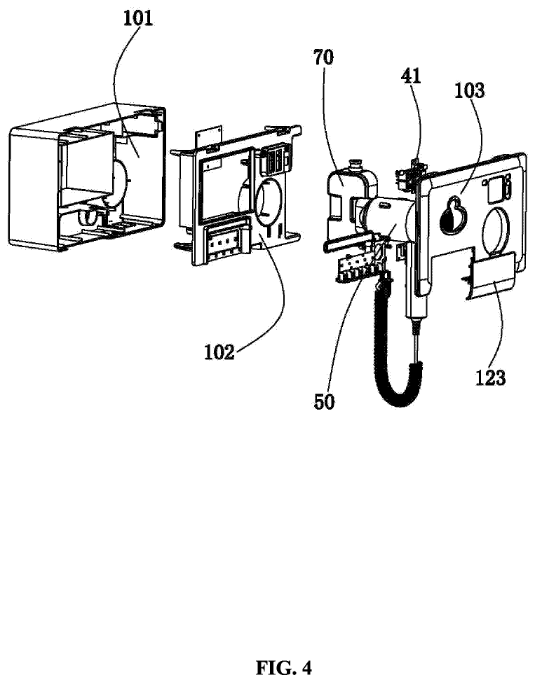

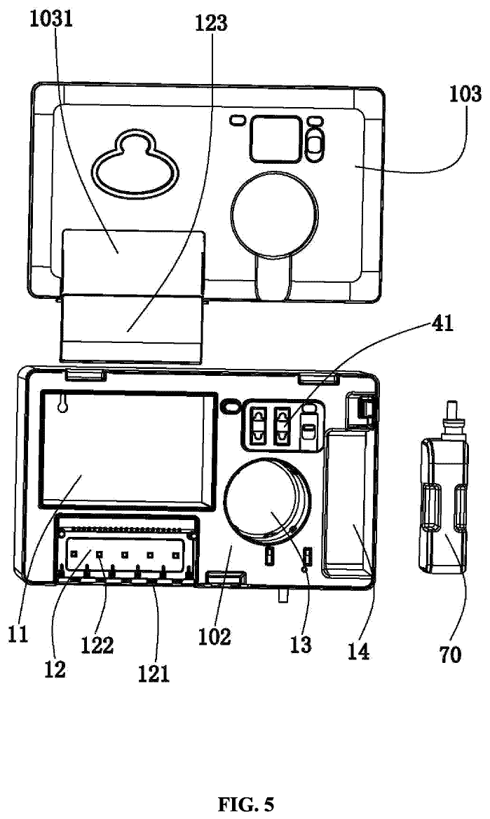

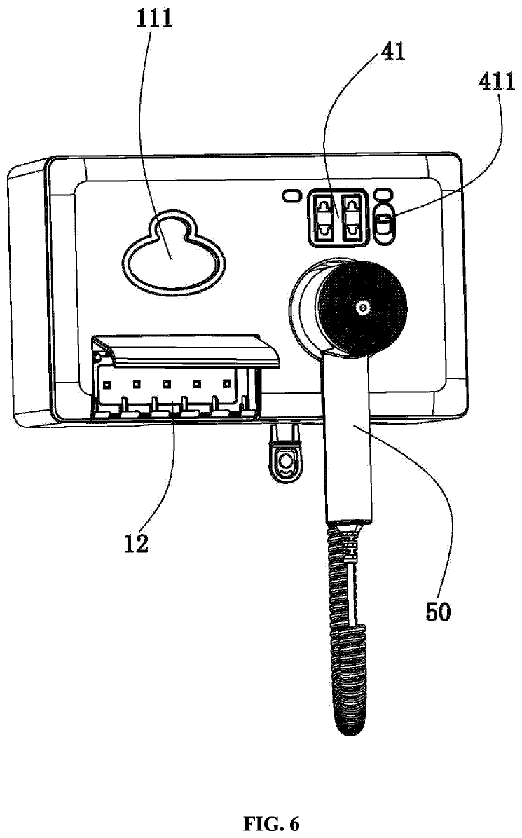

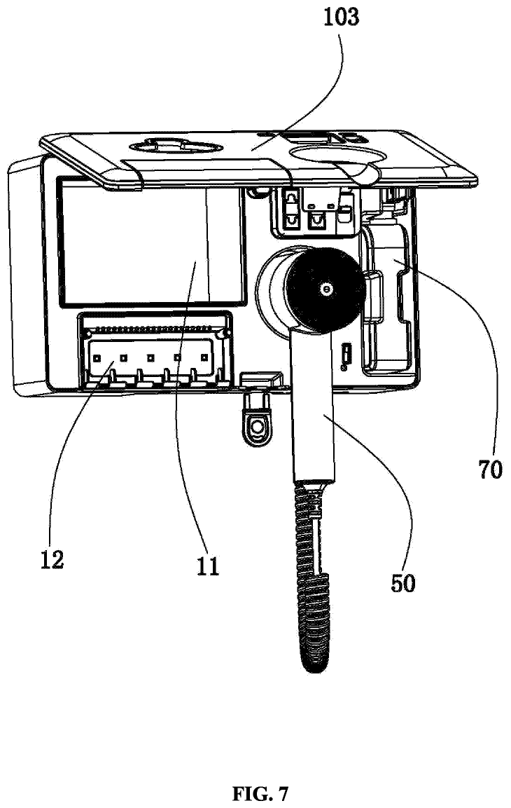

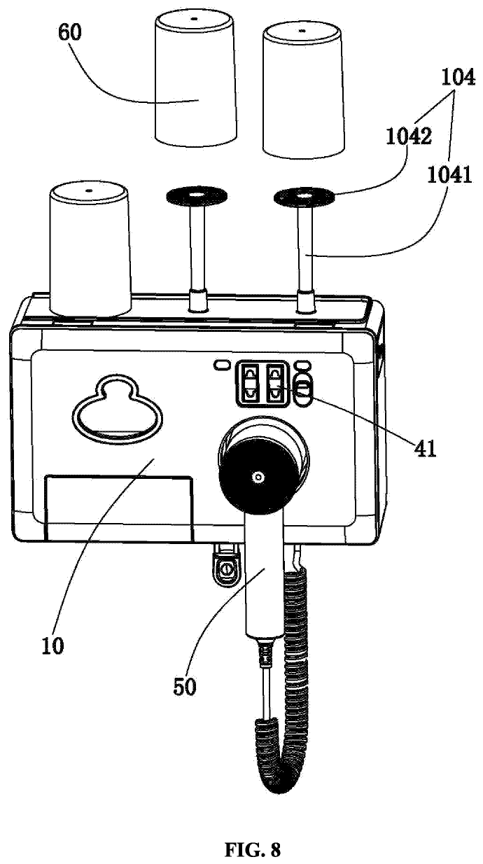

Referring to FIG. 1 to FIG. 8, a multifunctional storage box in an embodiment of the present disclosure includes a storage box body 10 which is provided with a first storage cavity 11 used for placing paper towels, a second storage cavity 12 used for placing toothbrushes, and a third storage cavity 13 used for placing a hair dryer. Usually, the storage box body is of a cuboid structure.

The first storage cavity 11 is provided with a first window 111 used for taking out paper towels; and the first window 111 is formed in a front side of the first storage cavity 11.

The second storage cavity 12 is provided with several hanging parts 121 used for hanging toothbrushes; and a lower side of the second storage cavity 12 is provided with an avoiding opening for avoiding toothbrushes.

A front side of the third storage cavity 13 is provided with a second window 131 used for allowing an air outlet tube to enter.

In this way, the first storage cavity 11, the second storage cavity 12 and the third storage cavity 13 are arranged on the storage box to gather and store a tissue box, toothbrushes and a hair dryer on one storage box, so that the storage box has the advantage of reasonable and ingenious storage, and it is convenient to store the toothbrushes, the hair dryer and paper towels in a bathroom.

Specifically, the first storage cavity 11 is arranged at an upper side of the second storage cavity 12; and the third storage cavity 13 is arranged at a right side of the first storage cavity 11. Further, a right side of the third storage cavity 13 is also provided with a fourth storage cavity 14 used for placing an atomizer; and a fog outlet 141 is formed in the fourth storage cavity 14. During application, the fog outlet 141 can be arranged on a right side surface of the storage box body 10. The atomizer can atomize perfume, so that the storage box of the present disclosure also has a perfume atomization function. Of course, the fourth storage cavity 14 can also be a storage cavity for storing perfume.

Preferably, an ultraviolet radiator 122 used for disinfecting a toothbrush is arranged in the second storage cavity 12. Usually, a plurality of ultraviolet radiators 122 are arranged in the second storage cavity 12 at intervals. In this way, the toothbrush is cleaned, and is made to be cleaner and hygienic.

Further, the storage box body 10 includes a bottom shell 101, a middle plate 102 and a surface cover 103. Usually, the bottom shell 101, the middle plate 102 and the surface cover 103 are all made of plastic. The middle plate 102 is arranged on the bottom shell 101, and is provided with an inner cavity; the surface cover 103 is rotatably connected to an upper end of the bottom shell 101, and is located at a front side of the middle plate 102.

A PCB 40 is arranged in the inner cavity; and the PCB 40 is provided with a power line connected to a household power supply and a power supply socket 41 for supplying power. Preferably, the power supply socket 41 is arranged above the third storage cavity 13. Preferably, the PCB 40 can also be provided with a toggle switch 411 for powering off or powering on the power supply socket 41. The aforementioned ultraviolet radiator 122 is electrically connected to the PCB 40.

Further, the multifunctional storage box also includes a hair dryer 50 placed in the third storage cavity 13. The PCB 40 is electrically connected to the hair dryer 50. It should be added that the aforementioned atomizer can be an atomizer for atomizing perfume through an ultrasonic atomization sheet. Furthermore, the PCB 40 is preferably in controlled connection to the atomizer. In addition, it should be noted that a turn-on/turn-off module for controlling the atomizer is correspondingly arranged on the PCB 40, and the atomizer is connected to the PCB 40 through a wire. This is the prior art, so descriptions thereof are omitted here.

Further, a wall-mount hole 1011 is formed in a rear side surface of the bottom shell 101, so that the storage box body 10 can be hung and mounted on a wall. A wire avoiding slot 1012 for allowing the power line to pass is also formed in the rear side surface of the bottom shell 101.

The second storage cavity 12 is formed between the middle plate 102 and the surface cover 103, and the surface cover 103 is provided with an avoiding window 1031 corresponding to the second storage cavity 12. The avoiding window 1031 is provided with a cover plate 123 in an opening and closing manner. Usually, the cover plate 123 is rotatably connected to the avoiding window 1031 of the surface cover 103. A toothbrush can be placed or picked by correspondingly opening the surface cover 103 only. By the arrangement of the cover plate 123, the toothbrush can be effectively avoided from being polluted.

Preferably, several supporting columns 104 used for placing a tooth mug 60 can also be arranged on an upper side surface of the storage box body 10. Thus, a user can stably place the tooth mug 60 on the upper side surface of the storage box body 10, and the tooth mug will not slide off, so that the storage function of the storage box of the present disclosure is further enhanced. Specifically, each supporting column 104 includes a vertical column 1041 and a supporting disk 1042 arranged on the vertical column 1041.

The design key of the present disclosure is mainly as follows: A storage box can be designed to place paper towels, toothbrushes and a hair dryer, and is particularly suitable for a washroom. Meanwhile, the storage box has the advantages of reasonable layout and space saving due to reasonable arrangement of a first storage cavity, a second storage cavity and a third storage cavity.

Secondly, the fourth storage cavity for storing the atomizer is also included, which further enriches the function of the storage box. The power supply socket is also included to improve the practicability of the storage box of the present disclosure.

Furthermore, the ultraviolet radiator is arranged in the second storage cavity, which further ensures the cleanliness and sanitation of personal hygiene products.

The above descriptions are only preferred embodiments of the present disclosure, and do not limit the technical scope of the present disclosure. Therefore, any minor changes, equivalent changes and modifications made to the above embodiments according to the technical essence of the present disclosure still fall within the scope of the technical solutions of the present disclosure.

What is claimed is:

1. A multifunctional storage box, comprising a storage box body which is provided with a first storage cavity used for placing paper towels, a second storage cavity used for placing toothbrushes, and a third storage cavity used for placing a hair dryer, wherein
the first storage cavity is provided with a first window used for taking out paper towels; the first window is formed in a front side of the first storage cavity;
the second storage cavity is provided with several hanging parts used for hanging toothbrushes; a lower side of the second storage cavity is provided with an avoiding opening for avoiding toothbrushes;
a front side of the third storage cavity is provided with a second window used for allowing an air outlet tube to enter.

2. The multifunctional storage box according to claim 1, wherein the first storage cavity is arranged at an upper side of the second storage cavity; and the third storage cavity is arranged at a right side of the first storage cavity.

3. The multifunctional storage box according to claim 2, wherein the storage box body is further provided with a fourth storage cavity used for placing an atomizer; and a fog outlet is formed in the fourth storage cavity.

4. The multifunctional storage box according to claim 2, wherein the storage box body comprises a bottom shell, a middle plate and a surface cover; the middle plate is arranged on the bottom shell, and is provided with an inner cavity; the surface cover is rotatably connected to the bottom shell, and is located at a front side of the middle plate;
a Printed Circuit Board (PCB) is arranged in the inner cavity; and the PCB is provided with a power line connected to a household power supply and a power supply socket for supplying power.

5. The multifunctional storage box according to claim 4, further comprising a hair dryer placed in the third storage cavity, wherein the PCB is electrically connected to the hair dryer.

6. The multifunctional storage box according to claim 4, wherein a rear side of the bottom shell is provided with a wall-mount hole.

7. The multifunctional storage box according to claim 4, wherein the bottom shell, the middle plate and the surface cover are all made of plastic.

8. The multifunctional storage box according to claim 4, wherein the second storage cavity is formed between the middle plate and the surface cover; the surface cover is provided with an avoiding window corresponding to the second storage cavity; and the avoiding window is provided with a cover plate in an opening and closing manner.

9. The multifunctional storage box according to claim 4, wherein an ultraviolet radiator used for disinfecting a toothbrush is arranged in the second storage cavity, and the ultraviolet radiator is electrically connected to the PCB.
